(19) United States
(12) Patent Application Publication
(10) Pub. No.: US 2020/0139358 A1
(43) Pub. Date: May 7, 2020

(54) AROMATIC TRANSALKYLATION CATALYSTS

(71) Applicant: Clariant International Ltd, Muttenz (CH)

(72) Inventors: Malati RAGHUNATH, Palo Alto, CA (US); Matthew GREANEY, Hayward, CA (US); Aaron B. MILLER, San Ramon, CA (US); Claus G. LUGMAIR, Santa Cruz, CA (US); Anthony F. VOLPE, Santa Clara, CA (US)

(21) Appl. No.: 16/592,844

(22) Filed: Oct. 4, 2019

Related U.S. Application Data
(60) Provisional application No. 62/755,641, filed on Nov. 5, 2018.

(51) Int. Cl. B01J 29/78; B01J 23/22; B01J 35/10; B01J 6/00; C07C 2/66; B01J 37/00; B01J 23/42

(52) U.S. Cl. CPC B01J 29/7869; B01J 23/22; B01J 35/1019; B01J 23/42; C07C 2/66; B01J 37/0009; B01J 6/001

(57) ABSTRACT

The present disclosure relates to zeolite-containing catalysts useful in the transalkylation of aromatic hydrocarbons, such as the isomerization of ethylbenzene, to methods for making such catalysts, and to methods for aromatic transalkylation with such catalysts. One aspect of the disclosure provides an aromatic transalkylation catalyst that includes one or more zeolites, an inorganic binder, a transition metal catalyst, and vanadium.

### BACKGROUND OF THE DISCLOSURE

### Field of the Disclosure

This disclosure relates generally to solid catalyst materials. More particularly, the present disclosure relates to zeolite-containing catalysts useful in the transalkylation of aromatic hydrocarbons, such as the isomerization of ethylbenzene, to methods for making such catalysts, and to methods for aromatic transalkylation with such catalysts.

### Technical Background

Xylenes, i.e., including para-xylene (pX), meta-xylene, ortho-xylene (oX) and various combinations thereof, are important chemicals with wide and varied applications. Of the three isomers, para-xylene is particularly valuable (e.g., as a feedstock for the production of polyester fibers, PET resins, etc.), but is obtained in relatively small amounts via aromatics refining processes.

The ratio and/or amounts of C₈ isomers (including para-xylene) obtained from a refining process can be adjusted by catalyzed transalkylation reactions such as ethylbenzene isomerization and toluene disproportionation. These transalkylation catalysts are typically solid, porous materials comprising one or more zeolitic materials. Such catalysts are conventionally made by calcining mixtures of one or more zeolites and a silicon- or aluminum-comprising binder material and impregnating the calcined material with a transition metal.

However, these catalysts, when reacted with alkylaromatic hydrocarbons under conditions sufficient to produce a desirable, near-equilibrium distribution of xylenes (i.e., through ethylbenzene isomerization or xylene redistribution), often also degrade a significant portion of the desired C₈ aromatic hydrocarbons (e.g., through disproportionation or dealkylation of xylenes and/or ethylbenzene).

Accordingly, there remains a need for improved aromatic transalkylation catalysts and methods of using them.

### SUMMARY OF THE DISCLOSURE

One aspect of the disclosure is a calcined aromatic transalkylation catalyst composition comprising:
vanadium, present in the composition in an amount within the range of 0.01 wt. % to 8 wt. %, calculated as elemental metal;
one or more zeolites, present in the composition in a combined amount within the range of 1 wt. % to 80 wt. %;
an inorganic binder, present in the composition in an amount within the range of 10 wt. % to 99 wt. %; and
a transition metal catalyst, present in the composition in an amount up to 8 wt. %, calculated as elemental metal.

Another aspect of the disclosure is A method for transalkylating aromatic hydrocarbons, the method comprising contacting an aromatic hydrocarbon feed with A catalyst composition as described herein. In certain embodiments, the aromatic transalkylation is toluene disproportionation. In other embodiments, the aromatic transalkylation is ethylbenzene isomerization.

Other aspects of the disclosure will be apparent to the person of ordinary skill in the art based on the description below.

### BRIEF DESCRIPTION OF THE DRAWINGS

FIG. 1 is a set of graphs showing para-xylene production relative to total xylene production (pX/X) (left) and C₈ aromatic ring loss (C8RL) (right) data for a variety of catalysts described herein, as a function of ethylbenzene (EB) conversion.

FIG. 2 is a set of graphs showing C₈ aromatic ring loss (C8RL) (left) and para-xylene production relative to total xylene production (pX/X) (right) data for a variety of catalysts described herein, as a function of ethylbenzene (EB) conversion.

FIG. 3 is a set of graphs showing C₈ aromatic ring loss (C8RL) (left) and para-xylene production relative to total xylene production (pX/X) (right) data for a variety of catalysts described herein, as a function of ethylbenzene (EB) conversion.

FIG. 4 is a set of graphs showing C₈ aromatic ring loss (C8RL) (left) and para-xylene production relative to total xylene production (pX/X) (right) data for a variety of catalysts described herein, as a function of ethylbenzene (EB) conversion.

FIG. 5 is a set of graphs showing C₈ aromatic ring loss (C8RL) (left) and para-xylene production relative to total xylene production (pX/X) (right) data for a variety of catalysts described herein, as a function of ethylbenzene (EB) conversion.

### DETAILED DESCRIPTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within limits of precision typical in the art.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The disclosure relates to calcined aromatic transalkylation catalyst compositions that include one or more zeolites, an inorganic binder, a transition metal catalyst, and vanadium. In various aspects and embodiments of the compositions as otherwise described herein, the one or more zeolites and binder together form a porous structure, with the transition metal and the vanadium being substantially disposed at the surface of the porous structure. In other aspects and embodiments, the one or more zeolites, inorganic binder, and vanadium together form a porous structure, with the transition metal catalyst being substantially disposed at the surface of the porous structure. In certain such aspects and embodiments, the vanadium can be distributed throughout the structure of one or more of the zeolites, or the vanadium can be substantially disposed at the surface of one or more of the zeolites. As demonstrated herein, in various embodiments such catalysts can exhibit improved activity and/or selectivity for xylenes, minimizing C₈ aromatic hydrocarbon loss relative to conventional catalysts.

One aspect of the disclosure is an aromatic transalkylation catalyst composition. The catalyst composition includes vanadium, present in an amount within the range of 0.01 wt. % to 8 wt. %, calculated as elemental metal. The catalyst composition also includes one or more zeolites, present in a combined amount within the range of 1 wt. % to 80 wt. %, and an inorganic binder, present in an amount within the range of 10 wt. % to 98 wt. %. The catalyst composition may also include a transition metal catalyst, present in an amount up to 8 wt. %, calculated as elemental metal.

As noted above, in various aspects, vanadium is present in an amount within the range of 0.01 wt. % to 8 wt. %, calculated as elemental metal. Vanadium can be present, for example, in a variety of forms such as, for example, oxides and oxo compounds (e.g., vanadyl or vanadate compounds). As used herein, the term “vanadium” includes monometallic species (e.g., monometallic vanadyl) or localized domains (e.g., oxide clusters) that may be an integral part of a larger, chemically bonded structure—that is, dispersed throughout a composition. Vanadium may be entirely incorporated into a composition (e.g., monometallic species, or nanometer- or micron-sized domains dispersed throughout a structure), or may be, in part, spatially separated as separate domains wihtin a composition (e.g., monomeric species or spheroidal particles fused to the surface of a zeolite and/or binder structure). The present inventors have determined that vanadium can provide a variety of advantageous properties to the catalyst compositions, as described in more detail herein.

In certain embodiments as otherwise described herein, vanadium is present in the catalyst composition in an amount within the range of 0.01 wt. % to 7 wt. %, or 0.01 wt. % to 6 wt. %, or 0.01 wt. % to 5 wt. %, or 0.01 wt. % to 4 wt. %, or 0.01 wt. % to 3 wt. %, or 0.01 wt. % to 2 wt. %, or 0.025 wt. % to 8 wt. %, or 0.05 wt. % to 8 wt. %, or 0.1 wt. % to 8 wt. %, or 0.2 wt. % to 8 wt. %, or 0.3 wt. % to 8 wt. %, or 0.4 wt. % to 8 wt. %, or 0.5 wt. % to 8 wt. %, or 0.75 wt. % to 8 wt. %, or 1 wt. % to 8 wt. %, or 1.5 wt. % to 8 wt. %, or 2 wt. % to 8 wt. %, or 3 wt. % to 8 wt. %, or 4 wt. % to 8 wt. %, or 0.025 wt. % to 5 wt. %, or 0.05 wt. % to 4 wt. %, or 0.075 wt. % to 3 wt. %, or 0.1 wt. % to 2 wt. %, calculated as elemental metal.

As noted above, in various aspects, the catalyst composition includes one or more zeolites, present in a combined within the range of 1 wt. % to 80 wt. %. For example, in certain embodiments as otherwise described herein, the catalyst composition includes a single zeolite. In another example, in certain embodiments as otherwise described herein, the catalyst composition includes two zeolites, e.g., both of the same type, or of two different types. In certain desirable embodiments of the catalyst compositions described herein, the one or more zeolites have a structure (i.e., a pore size and channel structure) well-suited for aromatic transalkylation substrates such as, for example, ethylbenzene and/or xylenes. In certain embodiments as otherwise described herein, the one or more zeolites includes (or are) a zeolite of an MTW, MFI, ERI, MEL, MTT, FER, MWW, MOR, MAZ, BEA, FAU, or VFI structure type. For example, in certain such embodiments, the one or more zeolites includes a zeolite having an MTW structure type, or each of the one or more zeolites is of an MTW structure type. In certain embodiments as otherwise described herein, the catalyst composition includes a single zeolite, e.g., a zeolite having an MTW structure type.

The person of ordinary skill in the art will appreciate that the zeolites are materials that are substantially formed from silicon and aluminum oxides, with various ratios and various structural characteristics (e.g., pores of particular sizes) defining the type of zeolite. The person of ordinary skill in the art will appreciate that zeolites of any certain structural type may have a wide range of silica-to-alumina ratios (SARs). For example, ZSM-5, an MFI-type zeolite, can comprise silica and alumina in a ratio ranging from 12:1 (i.e., an SAR of 12) to infinity (i.e., essentially silica). In certain embodiments as otherwise described herein, the SAR of one or more zeolites of the catalyst composition is within the range of 15 to 200. For example, in certain such embodiments, the SAR of each of the one or more zeolites of the catalyst composition is within the range of 25 to 200, or 50 to 200, or 75 to 200, or 100 to 200, or 25 to 175, or 25 to 150, or 25 to 125, or 25 to 100, or 50 to 175, or 50 to 150, or 75 to 125. In one example, in certain embodiments as otherwise described herein, the catalyst composition includes an MTW-type zeolite having an SAR within the range of 50 to 150, or 75 to 125.

In certain embodiments as otherwise described herein, a relatively small amount (e.g., no more than 20%, or no more than 15%, or no more than 10%, or no more than 5%) of the silica and alumina tetrahedra of a zeolite have been exchanged for another species (e.g., phosphorus, nitrogen, or vanadium); such materials will nonetheless be considered zeolites of a particular type if they have a similar pore size and channel structure as compared to the base zeolite type. For example, in certain such embodiments, the catalyst composition includes an MTW-type zeolite, in which up to 10% (e.g., 0.5% to 2%, or 1% to 5%) of the silica and alumina tetrahedra of the zeolite are exchanged for vanadium.

In certain embodiments as otherwise described herein, the one or more zeolites are present in the catalyst composition in a combined amount within the range of 1 wt. % to 70 wt. %, or 1 wt. % to 60 wt. %, or 1 wt. % to 50 wt. %, or 1 wt. % to 40 wt. %, or 1 wt. % to 30 wt. %, or 1 wt. % to 25 wt. %, or 1 wt. % to 20 wt. %, or 1 wt. % to 15 wt. %, or 1 wt. % to 10 wt. %, or 2.5 wt. % to 50 wt. %, or 2.5 wt. % to 45 wt. %, or 2.5 wt. % to 40 wt. %, or 5 wt. % to 35 wt. %, or 5 wt. % to 30 wt. %, or 5 wt. % to 25 wt. %, or 5 wt. % to 20 wt. %, or 10 wt % to 70 wt %, or 10 wt % to 60 wt %, or 10 wt % to 50 wt %.

As noted above, in various aspects, the catalyst composition includes an inorganic binder, present in an amount within the range of 10 wt. % to 99 wt. %. In certain embodiments as otherwise described herein, the inorganic binder is silica, alumina, or a mixture thereof. As used herein, the terms “alumina” and “silica” include aluminum oxide and silicon oxide, respectively. As used herein, the term “oxide,” including, e.g., “mixed oxide,” “vanadium oxide,” “aluminum oxide,” etc., includes oxides in all forms and crystalline phases. For example, “aluminum oxide” includes Al₂O₃, Al₂Oₓ wherein x is within the range of 1 to 3, etc. Unless otherwise indicated, regardless of the actual stoichiometry of the oxide, oxides are calculated as the most stable oxide for purposes of weight percent determinations. For example, the person of ordinary skill in the art will appreciate that a non-stoichiometric oxide of aluminum oxide, or even another form of aluminum, may still be calculated as Al₂O₃ for purposes of weight percent determinations. Moreover, unless otherwise indicated, the compositions are described on an as-calcined basis.

In certain embodiments as otherwise described herein, the inorganic binder comprises alumina. In certain such embodiments, at least 50 wt. %, or at least 75 wt. %, or at least 90 wt. %, or at least 95 wt. % of the inorganic binder is alumina. For example, in certain embodiments as otherwise described herein, the inorganic binder consists essentially of alumina, or is alumina.

In certain embodiments as otherwise described herein, the inorganic binder is present in the catalyst composition in an amount within the range of 20 wt. % to 99 wt. %, or 30 wt. % to 99 wt. %, or 40 wt. % to 99 wt. %, or 50 wt. % to 99 wt. %, or 60 wt. % to 99 wt. %, or 65 wt. % to 99 wt. %, or 70 wt. % to 99 wt. %, or 75 wt. % to 99 wt. %, or 80 wt. % to 99 wt. %, or 85 wt. % to 99 wt. %, or 90 wt. % to 99 wt. %, or 60 wt. % to 98 wt. %, or 65 wt. % to 98 wt. %, or 70 wt. % to 98 wt. %, or 75 wt. % to 95 wt. %, or 80 wt. % to 95 wt. %.

As noted above, in various aspects, the catalyst composition can include a transition metal catalyst in an amount up to 8 wt. %, calculated as elemental metal. In certain embodiments as otherwise described herein, the transition metal catalyst is an element of groups 6-10 of the periodic table of the elements (e.g., chromium, manganese, iron, cobalt, nickel, molybdenum, rhodium, palladium, or platinum). For example, in certain such embodiments, the transition metal catalyst is platinum. The person of ordinary skill in the art will appreciate that the transition metal catalyst can be present in a variety of forms such as, for example, oxides, halides, silicates, aluminates, etc., or a mixture thereof. However, in certain embodiments as otherwise described herein, the transition metal is present in the catalyst composition in a substantially reduced form. For example, in certain such embodiments, at least 50 mol. % at least 75 mol. %, or at least 90 mol. % of the transition metal is present in the zero oxidation state.

In certain embodiments as otherwise described herein, the transition metal catalyst is present in the catalyst composition an amount within the range of 0.01 wt. % to 7 wt. %, or 0.01 wt. % to 6 wt. %, or 0.01 wt. % to 5 wt. %, or 0.01 wt. % to 4 wt. %, or 0.01 wt. % to 3 wt. %, or 0.01 wt. % to 2 wt. %, or 0.05 wt. % to 8 wt. %, or 0.1 wt. % to 8 wt. %, or 0.15 wt. % to 8 wt. %, or 0.2 wt. % to 8 wt. %, or 0.25 wt. % to 8 wt. %, or 0.5 wt. % to 8 wt. %, or 0.025 wt. % to 7 wt. %, or 0.05 wt. % to 6 wt. %, or 0.075 wt. % to 5 wt. %, or 0.1 wt. % to 4 wt. %, or 0.15 wt. % to 3 wt. %, or 0.2 wt. % to 2 wt. %, or 0.25 wt. % to 1 wt. %, calculated as elemental metal.

In certain embodiments as otherwise described herein, the catalyst composition includes vanadium, present in an amount within the range of 0.01 wt. % to 6 wt. % (e.g., 0.01 wt. % to 4 wt. %, or 0.01 wt. % to 3 wt. %), one or more zeolites, present in an amount within the range of 1 wt. % to 30 wt. % (e.g., 1 wt. % to 20 wt. %, or 1 wt. % to 15 wt. %), an inorganic binder, present in an amount within the range of 70 wt. % to 99 wt. % (e.g., 80 wt. % to 99 wt. %, or 85 wt. % to 99 wt. %), and a transition metal catalyst, present in an amount within the range of 0.01 wt. % to 2 wt. % (e.g., 0.01 wt. % to 1.5 wt. %, or 0.01 wt. % to 1 wt. %). In certain such embodiments, the one or more zeolites includes a single MTW-type zeolite. In certain such embodiments, the inorganic binder is alumina. In certain such embodiments, the transition metal catalyst is platinum. Advantageously, the present inventors have determined that such compositions can be especially useful, for example, as an ethylbenzene isomerization catalyst, as described in more detail below.

The person of ordinary skill in the art will appreciate that other components may be present in the compositions as described herein. However, in certain embodiments of the compositions as otherwise described herein, one or more zeolites, inorganic binder, transition metal catalyst (e.g., platinum), and vanadium make up at least 70 wt. %, or at least 75 wt. %, or at least 80 wt. %, or at least 85 wt. %, or at least 90 wt. %, or at least 95 wt. % of the composition.

In certain embodiments of the compositions as otherwise described herein, one or more of the zeolites present in the catalyst composition comprise at least 50 wt. % of the vanadium present in the composition. For example, in certain such embodiments, one or more of the zeolites present in the composition comprise at least 55 wt. %, or at least 60 wt. %, or at least 65 wt. %, or at least 70 wt. %, or at least 75 wt. %, or at least 80 wt. %, or at least 85 wt. %, or at least 90 wt. %, or at least 95 wt. %, or at least 99 wt. % of the vanadium present in the composition. In certain such embodiments, the vanadium is distributed throughout one or more of the zeolites present in the composition (e.g., distributed substantially evenly throughout one or more of the zeolites). Such materials can be made, for example, by first making a silica-alumina gel further comprising vanadium, and hydrothermally treating the gel to provide a zeolite (i.e., sol-gel processing). In other such embodiments, the vanadium is localized on the surface of one or more of the zeolites present in the composition. Such materials can be made, for example, by impregnating one or more zeolites with vanadium (e.g., before making a material including the one or more zeolites and the binder). The person of ordinary skill in the art will appreciate that a species (e.g., atoms, particles, etc.) “localized on a surface” includes species chemically bound to an atom of a surface and species that are themselves part of the surface (e.g., by exchange with surface atoms, intercalation, etc.). Species “localized on a surface” have a substantially higher concentration (e.g., at least 100% higher) at the surface of the material (including a surface of an internal pore) than in the interior of the material. The person of ordinary skill in the art will further appreciate that the “surface” of a composition does not consist solely of the outermost layer of atoms of a composition, but rather includes, e.g., the outermost 50 nm, 100 nm, 250 nm, 500 nm, 750 nm, or even 1 μm of the surface depth of the composition.

For example, in certain embodiments as otherwise described herein, at least 50 wt. %, or at least 55 wt. %, or at least 60 wt. %, or at least 65 wt. %, or at least 70 wt. %, or at least 75 wt. %, or at least 80 wt. %, or at least 85 wt. %, or at least 90 wt. %, or at least 95 wt. %, or at least 99 wt. % of the vanadium present in the composition is localized on the surface of one or more of the zeolites present in the composition.

Desirably, the compositions of the disclosure have a porous structure. As the person of ordinary skill in the art will appreciate, the zeolites themselves will have nano-scale pores (e.g., of differing sizes and shapes depending on the particular zeolite), but it is also desirable for the material to have a larger pore structure as well, for example, to allow for reactants to have a large area of contact with the one or more zeolites and the transition metal catalyst.

In certain embodiments of the compositions as otherwise described herein, one or more zeolites and inorganic binder together form a porous structure (i.e., with the one or more zeolites and the inorganic binder distributed evenly throughout the material). In certain such embodiments, the transition metal catalyst is localized on the surface of the porous structure comprising the one or more zeolites and inorganic binder. Such materials can be made, for example, by first making a porous material including the one or more zeolites and the inorganic binder (e.g., by extrusion and calcining), then impregnating the porous material with the transition metal catalyst or a precursor therefor. For example, in certain embodiments as otherwise described herein, at least 50 wt. %, or at least 55 wt. %, or at least 60 wt. %, or at least 65 wt. %, or at least 70 wt. %, or at least 75 wt. %, or at least 80 wt. %, or at least 85 wt. %, or at least 90 wt. %, or at least 95 wt. %, or at least 99 wt. % of the transition metal catalyst present in the composition is localized on the surface of a porous structure comprising the one or more zeolites and the binder. In certain such embodiments, one or more of the zeolites forming the porous structure comprises at least 50 wt. % (e.g., at least 70 wt. %, or at least 90 wt. %) of the vanadium present in the composition.

In another example, in certain embodiments as otherwise described herein, each of the transition metal catalyst and the vanadium is localized on the surface of a porous structure comprising the one or more zeolites and inorganic binder. Such materials can be made, for example, by first making a porous material including the one or more zeolites and the inorganic binder (e.g., by extrusion and calcining), then impregnating the porous material with the vanadium and the transition metal catalyst or a precursor therefor. For example, in certain embodiments as otherwise described herein, at least 50 wt. %, or at least 55 wt. %, or at least 60 wt. %, or at least 65 wt. %, or at least 70 wt. %, or at least 75 wt. %, or at least 80 wt. %, or at least 85 wt. %, or at least 90 wt. %, or at least 95 wt. %, or at least 99 wt. % of each of the transition metal catalyst and the vanadium present in the composition are localized on the surface of a porous structure comprising the one or more zeolites and the binder.

The person of ordinary skill in the art will appreciate that a wide variety of porosities can be useful with respect to the compositions described herein, and that porosity can be measured in a variety of ways. In certain embodiments as otherwise described herein, the composition has a surface area within the range of 100 m²/g to 500 m²/g, e.g., 125 m²/g to 500 m²/g, or 150 m²/g to 500 m²/g, or 175 m²/g to 500 m²/g, or 200 m²/g to 500 m²/g, or 225 m²/g to 500 m²/g, or 250 m²/g to 500 m²/g, or 125 m²/g to 475 m²/g, or 150 m²/g to 450 m²/g, or 175 m²/g to 425 m²/g, or 200 m²/g to 400 m²/g.

As the person of ordinary skill in the art will appreciate, and as described in more detail below, the compositions of the present disclosure can be prepared in a variety of manners. In certain desirable embodiments, a composition as otherwise described herein is in the form of a calcined extrudate. For example, in some embodiments of the compositions of the disclosure, the catalyst composition comprises a porous structure that is the calcined product of a formable mixture including a binder source and one or more zeolites, at least one zeolite comprising vanadium. In certain such embodiments, the catalyst composition is the calcined product of the porous structure impregnated with a transition metal catalyst or a source thereof. In other embodiments, the catalyst composition is comprises a porous structure that is the calcined product of a formable mixture including one or more zeolites and a binder source. In certain such embodiments, the catalyst composition is the calcined product of the porous structure impregnated with a vanadium source and a transition metal catalyst or a source thereof.

Another aspect of the disclosure is a method of preparing an aromatic transalkylation catalyst composition as described herein. The method includes providing a binder source and one or more zeolites, at least one zeolite comprising vanadium. The method includes forming a mixture comprising the one or more zeolites and the binder source (e.g., by extruding, tableting, or pelletizing), and calcining the formed mixture. A transition metal catalyst can be provided to the composition via one or more impregnation steps (i.e., after the material is formed, e.g., either before or after the calcining step). The person of ordinary skill in the art can provide the various source materials in amounts suitable to provide the desired amounts of vanadium, zeolite, binder, and transition metal catalyst.

In one example, in certain embodiments of the methods as otherwise described herein, providing at least one zeolite comprising vanadium includes reacting at least one zeolite with a vanadium solid. For example, in certain such embodiments, providing at least one zeolite comprising vanadium comprises reacting a zeolite (e.g., an MTW-type zeolite) with a vanadium solid (e.g., vanadium oxide) in the solid-state.

In another example, in certain embodiments of the methods as otherwise described herein, providing at least one zeolite comprising vanadium includes impregnating at least one zeolite with a vanadium source. For example, in certain such embodiments, providing at least one zeolite comprising vanadium comprises impregnating a zeolite (e.g., an MTW-type zeolite) with a vanadium source (e.g., vanadyl oxalate, vanadyl sulfate, or a mixture thereof).

In another example, in certain embodiments of the methods as otherwise described herein, providing at least one zeolite comprising vanadium includes hydrothermally treating a zeolite precursor comprising a vanadium source. For example, in certain such embodiments, providing at least one zeolite comprising vanadium comprises hydrothermally treating a zeolite precursor (e.g., an MTW-type zeolite precursor) comprising a vanadium source (e.g., vanadyl oxalate, vanadyl sulfate, or a mixture thereof).

Another aspect of the disclosure is a method of preparing an aromatic transalkylation catalyst composition as described herein. The method includes providing a binder source, one or more zeolites, and a vanadium source. The method includes forming a mixture comprising the binder source, the one or more zeolites, and the vanadium source (e.g., by extruding, tableting, or pelletizing), and calcining the formed mixture. A transition metal catalyst can be provided to the composition via one or more impregnation steps (i.e., after the material is formed, e.g., either before or after the calcining step). The person of ordinary skill in the art can provide the various source materials in amounts suitable to provide the desired amounts of vanadium, zeolite, binder, and transition metal catalyst.

Another aspect of the disclosure is a method of preparing an aromatic transalkylation catalyst composition as described herein. The method includes providing a binder source and one or more zeolites. The method includes forming a mixture comprising the binder source and the one or more zeolites (e.g., by extruding, tableting, or pelletizing), and calcining the formed mixture. A transition metal catalyst and a vanadium source can be provided to the composition via one or more impregnation steps (e.g., impregnating the calcined composition first with a vanadium source and second with a transition metal catalyst source, e.g., calcining the composition after each impregnation). The person of ordinary skill in the art can provide the various source materials in amounts suitable to provide the desired amounts of vanadium, zeolite, binder, and transition metal catalyst.

As described above, a vanadium solid can be used to provide vanadium to the compositions of the disclosure (e.g., via solid-state reaction with one or more zeolites). The vanadium solid may be, for example, vanadium oxide, or any other vanadium compound that reacts with a zeolite in the solid state to provide a vanadium-containing zeolite.

As described above, a vanadium source can be used to provide vanadium to the compositions of the disclosure (e.g., included in a zeolite precursor, impregnated into a zeolite, or included in a formable or formed mixture). The vanadium source may be, for example, vanadyl oxalate, vanadyl sulfate, a mixture thereof, or any other vanadium compound that can provide vanadium to the catalyst composition.

As noted above, the mixture includes one or more zeolites. Any of the zeolites or combinations thereof described above with respect to the compositions of the disclosure can be suitably used in the methods described herein (e.g., one or more zeolites having an MTW, MFI, ERI, MEL, MTT, FER, MWW, MOR, MAZ, BEA, FAU, or VFI structure type). As the person of ordinary skill in the art will appreciate, the processing conditions (e.g., of forming and calcining) can easily be chosen such that the zeolite structure itself does not appreciably change during the composition synthesis process. The zeolite(s) can be provided in the formable mixture in an amount sufficient to provide the desired amount of zeolite to the final calcined product (e.g., 1 wt. % to 80 wt. %, or any other amount described above).

As noted above, the mixture also includes a binder source. As the person of ordinary skill in the art will appreciate, the binder source is a may be any material that comprises or forms, upon calcination, an inorganic binder. Accordingly, in certain embodiments of the methods as otherwise described herein, the binder source is a silica source or an alumina source. The person of ordinary skill in the art can select a suitable binder source to provide any of the inorganic binders described above with respect to the compositions of the disclosure. In certain embodiments of the methods as otherwise described herein, the binder source itself is silica and/or alumina. In certain embodiments of the methods as otherwise described herein, the binder source is an aluminum hydroxide, i.e., Al(OH)ₓ(O)_{y}, in which X is at least 0.1. In various embodiments, the aluminum hydroxide is aluminum trihydroxide, e.g., bayerite, gibbsite, doyleite, or nordstrandite. In some embodiments, the aluminum hydroxide is aluminum oxide hydroxide, e.g., diaspore, boehmite, psuedoboehmite, or akdalaite. The binder source can be provided in the formable mixture in an amount sufficient to provide the desired amount of inorganic binder to the final calcined product (e.g., 10 wt. % to 99 wt. %, or any other amount described above).

The person of ordinary skill in the art will also appreciate that the forms of the mixture components (one or more zeolites, the binder source, and optionally, the vanadium source) may be varied and combined in a number of ways. The person of ordinary skill in the art will also appreciate that the order of addition of the mixture components may vary in a number of ways. In one example, the binder source and the one or more zeolites are mixed together before the vanadium source is added. In another example, the vanadium source, binder source, and one or more zeolites are added and mixed simultaneously.

The person of ordinary skill in the art will appreciate that other conventional materials can be included in the mixture, such as, for example, water, oil, or other materials to ad with mixing or forming (e.g., via extrusion).

The mixture may be mixed by a variety of methods, both manual and mechanical. In some embodiments, two or more components of the formable mixture are mixed mechanically. In some aspects, the mechanical mixing may be accomplished using, e.g., a planetary mixer, a spiral mixer, a stand mixer, screw extruder, etc.

The method of preparing an aromatic transalkylation catalyst composition also comprises forming the mixture. The person of ordinary skill in the art will appreciate that the mixture may be formed into a variety of shapes, e.g., extrudates, pellets, tablets, spheres, etc. A variety of means for forming such shapes are known in the art, e.g., extrusion, pelletizing, marumarizing, etc. In some embodiments, the mixture is formed by extrusion into an extrudate. The person of ordinary skill in the art will selected extrusion conditions to provide desired extrudate properties, e.g., shape size, etc.

The method also includes one or more calcining steps (e.g., after vanadium impregnation of one or more zeolites, after forming the mixture, after transition metal catalyst and/or vanadium impregnation of the composition). In some embodiments of the methods described herein, one or more calcination steps (e.g., each calcination step) may be performed at a temperature within the range of 200° C. to 700° C., e.g., 200° C. to 675° C., or 200° C. to 650° C., or 200° C. to 625° C., or 200° C. to 600° C., or 200° C. to 575° C., or 200° C. to 550° C., or 225° C. to 700° C., or 250° C. to 700° C., or 275° C. to 700° C., or 300° C. to 700° C., or 325° C. to 700° C., or 350° C. to 700° C., or 225° C. to 675° C., or 250° C. to 650° C., or 275° C. to 625° C., or 300° C. to 600° C., or 325° C. to 575° C., or 350° C. to 550° C.

In some embodiments of the methods described herein, one or more calcining steps (e.g., each calcining step) may be performed for a period of time within the range of 5 min. to 12 hr., e.g., 10 min. to 12 hr., or 15 min. to 12 hr., or 20 min. to 12 hr., or 30 min. to 12 hr., or 45 min. to 12 hr., or 1 hr. to 12 hr., or 1.5 hr. to 12 hr., or 2 hr. to 12 hr., or 5 min. to 11 hr., or 5 min. to 10 hr., or 5 min. to 9 hr., or 5 min. to 8 hr., or 5 min. to 7.5 hr., or 5 min. to 7 hr., or 5 min. to 6.5 hr., or 5 min. to 6 hr., or 5 min. to 5.5 hr., or 5 min. to 5 hr., or 30 min. to 11 hr., or 1 hr. to 10 hr., or 1.5 hr. to 9 hr., or 2 hr. to 8 hr.

In some embodiments of the methods described herein, a drying step is conducted before one or more calcining steps. In some embodiments, one or more drying steps (e.g., each drying step) may performed at a temperature within the range of 40° C. to 200° C., e.g., 60° C. to 200° C., or 80° C. to 200° C., or 100° C. to 200° C., or 40° C. to 180° C., or 40° C. to 160° C., or 40° C. to 140° C., or 60° C. to 180° C., or 80° C. to 160° C., or 100° C. to 140° C.

In some embodiments of the methods described herein, one or more drying steps (e.g., each drying step) may be performed for a period of time within the range of 4 hr. to 36 hr., e.g., 4 hr. to 30 hr., or 4 hr. to 24 hr., or 4 hr. to 22 hr., or 4 hr. to 20 hr., or 6 hr. to 36 hr., or 8 hr. to 36 hr., or 10 hr. to 36 hr., or 12 hr. to 36 hr., or 6 hr. to 30 hr., or 8 hr. to 24 hr., or 10 hr. to 22 hr., or 12 hr. to 20 hr., of a period of time of 10 hr., or 12 hr. or 14 hr. or 16 hr., or 18 hr., or 20 hr., or 22 hr.

As noted above, the transition metal catalyst can be provided to the composition via impregnation. Similarly, as noted above, in certain embodiments of the methods described herein, the vanadium is provided to the composition via impregnation. The person of ordinary skill in the art will use conventional methodologies to perform such impregnations, based on the disclosure herein. For example, incipient wetness methods can be used to perform the impregnation(s). Any desired impregnation process can be performed on the formed material, for example, before, after, or in between any calcining steps. The impregnated material may itself need to be calcined to provide the desired transition metal catalyst or vanadium.

A transition metal source can be used to provide the transition metal catalyst of the compositions of the disclosure. In certain embodiments of the methods as otherwise described herein, the transition metal catalyst source includes an element of groups 6-10 of the periodic table of the elements, e.g., chromium, manganese, iron, cobalt, nickel, molybdenum, rhodium, palladium, or platinum. For example, in certain embodiments of the methods as otherwise described herein, the transition metal source includes molybdenum. In other embodiments of the methods as otherwise described herein, the transition metal catalyst includes platinum. The person of ordinary skill in the art will appreciate that the transition metal catalyst source may comprise the transition metal in a variety of forms, e.g., inorganic and organic complexes, acids, oxides, halides, acetates, etc. In certain embodiments of the methods described herein, the transition metal catalyst source is an aqueous solution of the transition metal, e.g., an aqueous solution of ammonium hetpamolybdate or chloroplatinic acid. The person of ordinary skill in the art will select a pH and concentration for such solutions to provide desired impregnation properties (e.g., for incipient wetness impregnation). The transition metal catalyst source can be provided to an impregnated material in an amount sufficient to provide the desired amount of transition metal catalyst to the final calcined product (e.g., 0.01 wt. % to 8 wt. % on a metallic basis, or any other amount described above).

Another aspect of the disclosure is a catalyst composition prepared by a method as described herein. Advantageously, the present inventors have determined that vanadium-containing compositions, as otherwise described herein, can provide improved performance in transalkylation processes, as demonstrated in more detail below.

The compositions as otherwise described herein are especially useful in aromatic transalkylation reactions (i.e., conversion of alkylaromatic hydrocarbons), such as the disproportionation of toluene to form xylene, e.g., para-xylene, or the isomerization of ethylbenzene to form xylene, e.g., para-xylene. Accordingly, another aspect of the disclosure is a method for performing an aromatic transalkylation reaction including contacting an aromatic hydrocarbon feed with an aromatic transalkylation catalyst composition as described herein, under conditions sufficient to cause a transalkylation of the aromatic hydrocarbon.

In certain embodiments of the transalkylation methods as otherwise described herein, the aromatic hydrocarbon feed includes one or more alkylaromatic hydrocarbons of the formula C₆H₆₋ₓRₓ, where each R is independently an alkyl group such as CH₃, C₂H₅, C₃H₇, C₄H₉, or C₅H₁₁, for example, in a total wt. % of organic compounds in the feedstock of at least 10 wt. %, at least 20 wt. %, at least 50 wt. %, at least 75 wt. %, or even at least 90 wt. %.

For example, in certain embodiments of the transalkylation methods as otherwise described herein, the aromatic hydrocarbon feed contains ethylbenzene (e.g., in which at least 5 wt. %, at least 10 wt. %, or even at least 15 wt. % of the organic compounds of the feed are ethylbenzene). In certain embodiments of the transalkylation methods as otherwise described herein, when the aromatic hydrocarbon feed contains toluene, the reaction is an ethylbenzene isomerization to form xylene (e.g., para-xylene).

In another example, in certain embodiments of the transalkylation methods as otherwise described herein, the aromatic hydrocarbon feed contains toluene (e.g., in which at least 5 wt. %, at least 10 wt. %, at least 15 wt. %, over even at least 20 wt. % of the organic compounds of the feed are toluene). In certain such embodiments, the aromatic hydrocarbon feed includes at least 50 wt. %, at least 75 wt. %, or even at least 90 wt. % toluene (i.e., of the total organic content of the feed). In certain embodiments of the transalkylation methods as otherwise described herein, when the aromatic hydrocarbon feed contains toluene, the reaction is a disproportionation of toluene to form xylene.

The contacting of the aromatic hydrocarbon feed with the catalyst composition described herein can be conducted in a variety of ways familiar to the person of ordinary skill in the art. Conventional equipment and processes can be used in conjunction with the catalyst compositions of the disclosure to provide beneficial performance. Thus, the catalyst may be contained in one bed within a reactor vessel or divided up amount a plurality of beds within a reactor. The reaction system may contain one or more reaction vessels in series. The feed to the reaction zone can flow vertically upwards, or downwards through the catalyst bed in a typical plug flow reactor, or horizontally across the catalyst bed in a radial flow type reactor.

The transition metal catalyst is desirably in a substantially reduced form (e.g., as described above with respect to compositions of the disclosure). Accordingly, it can be desirable to treat the catalyst composition with hydrogen, for example, before contacting the catalyst composition with the aromatic hydrocarbon feed. Such treatment can be performed, for example, at a temperature in the range 250-400° C. in flowing hydrogen (e.g., GHSV in the range of 600-1200 h⁻¹) at a pressure in the range of 2-16 bar, for a time of at least 4 hours (e.g., 8-24 hours).

The contacting of the aromatic hydrocarbon feed with the catalyst composition can be performed using conventional methods. For example, the feed may be introduced into the reaction zone containing the catalyst composition at a constant rate, or alternatively, at a variable rate. The transalkylation can be conducted under at least partially vapor phase conditions.

In some aspects, the hydrocarbon feed may include any C₇, C₈, or C₉₊ aromatic hydrocarbon. As the person of ordinary skill in the art will appreciate, the aromatic hydrocarbon feed may include a number of combinations of C₇, C₈, and C₉₊ hydrocarbons. In some embodiments, the hydrocarbon feed includes toluene. In some embodiments, the hydrocarbon feed includes ethylbenzene.

In some embodiments, the aromatic hydrocarbon feed includes toluene in an amount within the range of 40 wt. % to 95 wt. %, e.g., 40 wt. % to 90 wt. %, or 45 wt. % to 90 wt. %, or 50 wt. % to 90 wt. %, or 55 wt. % to 90 wt. %, or 60 wt. % to 90 wt. %, or 65 wt. % to 90 wt. %, or 70 wt. % to 90 wt. %, or 75 wt. % to 85 wt. %.

In some embodiments, the aromatic hydrocarbon feed includes ethylbenzene in an amount within the range of 0.5 wt. % to 40 wt. %, e.g., 1 wt. % to 40 wt. %, or 2.5 wt. % to 37.5 wt. %, or 5 wt. % to 35 wt. %, or 7.5 wt. % to 32.5 wt. %, or 10 wt. % to 30 wt. %, or 10 wt. % to 27.5 wt. %, or 10 wt. % to 25 wt. %, or 10 wt. % to 22.5 wt. %, or 10 wt. % to 20 wt. %.

In some embodiments, the aromatic hydrocarbon feed is contacted with the provided transalkylation catalyst composition at a weight hourly space velocity of 1 h⁻¹ to 10 h⁻¹, e.g., in the range of 2-10 h⁻¹, or 3-10 h⁻¹, or 1-7.5 h⁻¹, or 2-7.5 h⁻¹, or 3-7.5 h⁻¹.

In some embodiments, the method of transalkylating aromatic hydrocarbons is carried out at a temperature within the range of 200° C. to 500° C., e.g., 250° C. to 500° C., or 300° C. to 500° C., or 350° C. to 500° C., or 200° C. to 450° C., or 250° C. to 450° C., or 300° C. to 450° C., or 350° C. to 450° C., or 200° C. to 400° C., or 250° C. to 400° C., or 300° C. to 400° C., or 200° C. to 350° C., or 250° C. to 350° C.

In some embodiments, the method of transalkylating aromatic hydrocarbons is carried out at a pressure within the range of 1-50 bar, e.g., 3-50 bar, or 6-50 bar, or 1-35 bar, or 3-35 bar, or 6-35 bar, 1-25 bar, or 3-25 bar, 6-25 bar.

### Examples

The Examples that follow are illustrative of specific embodiments of the invention, and various uses thereof. They are set forth for explanatory purposes only, and are not to be taken as limiting the invention.

### Example 1. Catalyst Preparation

0.90 g of an MTW-type zeolite having a silica-to-alumina ratio of 100 (i.e., SAR 100) and 10.25 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide comparative catalyst C1.

1.0 g of an MTW-type zeolite (SAR 100) and 0.1 g of V₂O₅ were ground in a mortar for 1 hr., dried at 120° C. for 3 hr., and calcined at 400° C. for 7 hr. to provide an MTW-type zeolite containing 5.6 wt. % vanadium. 0.90 g of the vanadium-containing zeolite and 10.25 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A1.

0.45 g of an MTW-type zeolite (SAR 100) and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide comparative catalyst C2. Catalysts C4 and C5 were prepared similarly (see Table 1, below).

2.0 g of an MTW-type zeolite (SAR 100) and 5 g of ammonium sulfate solution (0.05 M) were heated to 80° C. and mechanically mixed for 1 hr. The impregnated zeolites were dried at 120° C. for 4 hr. and calcined at 550° C. for 4 hr. to provide an MTW-type zeolite containing 1.2 wt. % sulfate. 0.90 g of the sulfate-containing zeolite and 10.25 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide comparative catalyst C3.

0.45 g of an MTW-type zeolite (SAR 100) and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.05 M vanadyl sulfate solution. The vanadium-impregnated extrudates were dried at 120° C. for 4 hr. and calcined at 500° C. Finally, the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A2. Catalysts A12 and A20-21 were prepared similarly (see Table 1, below; catalyst A20 was prepared with ammonium metavanadate solution; catalyst A21 was prepared with vanadium oxalate solution).

1.0 g of an MTW-type zeolite (SAR 100) and 5 g of vanadyl sulfate solution (0.05 M) were heated to 80° C. and mechanically mixed for 1 hr., washed, and filtered. The impregnated zeolites were dried at 120° C. for 4 hr. and calcined at 550° C. for 4 hr. to provide an MTW-type zeolite containing 1.2 wt. % vanadium. 0.45 g of the vanadium-containing zeolite and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A3. Catalysts A8-10 and A13 were prepared similarly (see Table 1, below).

1.0 g of an MTW-type zeolite (SAR 100) and 5 g of vanadyl oxalate solution (0.05 M) were heated to 80° C. and mechanically mixed for 1 hr., washed, and filtered. The impregnated zeolites were dried at 120° C. for 4 hr. and calcined at 550° C. for 4 hr. to provide an MTW-type zeolite containing 1.2 wt. % vanadium. 0.45 g of the vanadium-containing zeolite and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A4. Catalyst A14 was prepared similarly (see Table 1, below)

0.145 mL of sodium aluminate, 0.18 g of NaOH solution (30 wt. %) and 2.22 g of tetraethyl ammonium hydroxide (TEAOH) were dissolved in 7.75 mL of distilled water. 2.37 g of silica was then added, and the mixture was stirred for about 30 min. 0.5 mL of vanadyl sulfate solution (0.05 M) was added to the solution, and the mixture was stirred for 30 min. The mixture was transferred to a 23 mL Teflon-lined stainless steel autoclave and subjected to hydrothermal treatment at 160° C. for 4 days (static condition, autogenous pressure). After treatment, the crude product was recovered by filtration, washed thoroughly with hot distilled water, and dried at 60° C. The crude product was calcined under dry air flow, at 550° C. for 10 h to remove residual organic triethylamine cation. Finally, the resulting zeolite (Na-form) was ion-exchanged three times with ammonium nitrate at 60° C. for 1 hr., and calcined at 500° C. for 8 hr. to provide an H-form MTW-type zeolite (SAR 100) containing vanadium. 0.45 g of the vanadium-containing zeolite and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A5 having a vanadium loading of 0.05 wt. %. Catalyst A11 was prepared similarly (see Table 1, below).

Sodium aluminate and tetraethyl ammonium hydroxide (TEAOH) were dissolved in distilled water. Colloidal silica was then added, and the mixture was stirred for about 30 min., until visibly clear. 1 mL of vanadyl sulfate solution (0.05 M) was added to the solution, and the mixture was stirred for 30 min. The mixture was transferred to a 23 mL Teflon-lined stainless steel autoclave and subjected to hydrothermal treatment at 160° C. for 4 days (static condition, autogenous pressure). After treatment, the crude product was recovered by filtration, washed thoroughly with hot distilled water, and dried at 60° C. The crude product was calcined under dry air flow, at 550° C. for 10 h to remove residual organic triethylamine cation. Finally, the resulting zeolite (Na-form) was ion-exchanged three times with ammonium nitrate at 60° C. for 1 hr., and calcined at 500° C. for 8 hr. to provide an H-form MTW-type zeolite (SAR 100) containing 0.05 wt. % vanadium. 0.45 g of the vanadium-containing zeolite and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A6 having a vanadium loading of 0.1 wt. %. Catalysts A15 and A18-19 were prepared similarly (see Table 1, below; catalyst A18 was prepared with vanadyl oxalate solution).

Sodium aluminate and tetraethyl ammonium hydroxide (TEAOH) were dissolved in distilled water. Colloidal silica was then added, and the mixture was stirred for about 30 min., until visibly clear. 1.5 mL of vanadyl sulfate solution (0.05 M) was added to the solution, and the mixture was stirred for 30 min. The mixture was transferred to a 23 mL Teflon-lined stainless steel autoclave and subjected to hydrothermal treatment at 160° C. for 4 days (static condition, autogenous pressure). After treatment, the crude product was recovered by filtration, washed thoroughly with hot distilled water, and dried at 60° C. The crude product was calcined under dry air flow, at 550° C. for 10 h to remove residual organic triethylamine cation. Finally, the resulting zeolite (Na-form) was ion-exchanged three times with ammonium nitrate at 60° C. for 1 hr., and calcined at 500° C. for 8 hr. to provide an H-form MTW-type zeolite (SAR 100) containing 0.05 wt. % vanadium. 0.45 g of the vanadium-containing zeolite and 10.50 g of an alumina source were mechanically mixed for 5 min. 7.0 g of water, 2.5 g of concentrated acetic acid, and 0.5 g of lightweight paraffin extrusion oil were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. 1 g of the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A7 having a vanadium loading of 0.15 wt. %. Catalysts A16-17 were prepared similarly (see Table 1, below).

0.45 g of an MTW-type zeolite and 10.50 g of an alumina source were mechanically mixed for 5 min. 7.0 g of water, 2.5 g of concentrated acetic acid, 0.5 g of lightweight paraffin extrusion oil, and 0.6 mL of a 0.05 M vanadyl sulfate solution were manually mixed with the zeolite and alumina source until visibly homogeneous. The mixture was extruded using a hydraulic press at several kPsi into cylindrical extrudates having a 2-mm diameter and a 2-3-mm length, dried at 120° C. for 4 hr., and calcined at 500° C. for 4 hr. Finally, the calcined extrudates were impregnated by incipient wetness with 0.6 mL of a 0.3 wt. % platinum solution of chloroplatinic acid. The impregnated extrudates were agitated for 30 min., dried at 120° C. for 4 hr., and calcined at 500° C. for 4 hr. to provide catalyst A22.

TABLE 1
Catalyst Preparation

| | Vanadium | Zeolite | Zeolite | | Binder | Binder |
|---|---|---|---|---|---|---|
| Catalyst | (wt. %) | (wt. %) | Type | SAR | (wt. %) | Type |
| C1 | 0 | 10 | MTW | 100 | 90 | Alumina |
| C2 | 0 | 5 | MTW | 100 | 95 | Alumina |
| C3 | 0 | 5 | MTW | 100 | 95 | Alumina |
| C4 | 0 | 5 | MTW | 100 | 95 | Alumina |
| C5 | 0 | 5 | MTW | 100 | 95 | Alumina |
| A1 | 5.6 | 10 | MTW | 100 | 90 | Alumina |
| A2 | 1.2 | 5 | MTW | 100 | 95 | Alumina |
| A3 | 1.2 | 5 | MTW | 100 | 95 | Alumina |
| A4 | 1.2 | 5 | MTW | 100 | 95 | Alumina |
| A5 | 0.05 | 5 | MTW | 100 | 95 | Alumina |
| A6 | 0.1 | 5 | MTW | 100 | 95 | Alumina |
| A7 | 0.15 | 5 | MTW | 100 | 95 | Alumina |
| A8 | 1.2 | 10 | MTW | 100 | 90 | Alumina |
| A9 | 1.2 | 10 | MTW | 100 | 90 | Alumina |
| A10 | 1.2 | 5 | MTW | 100 | 95 | Alumina |
| A11 | 0.1 | 5 | MTW | 100 | 95 | Alumina |
| A12 | 1.2 | 5 | MTW | 100 | 95 | Alumina |
| A13 | 1.2 | 5 | MTW | 100 | 95 | Alumina |
| A14 | 1.2 | 5 | MTW | 100 | 95 | Alumina |
| A15 | 0.1 | 5 | MTW | 100 | 95 | Alumina |
| A16 | 0.2 | 5 | MTW | 100 | 95 | Alumina |
| A17 | 0.3 | 5 | MTW | 100 | 95 | Alumina |
| A18 | 0.1 | 5 | MTW | 100 | 95 | Alumina |
| A19 | 0.1 | 5 | MTW | 100 | 95 | Alumina |
| A20 | 1.2 | 5 | MTW | 100 | 95 | Alumina |
| A21 | 1.2 | 5 | MTW | 100 | 95 | Alumina |
| A22 | 1.2 | 5 | MTW | 100 | 95 | Alumina |

### Example 2. Aromatic Transalkylation

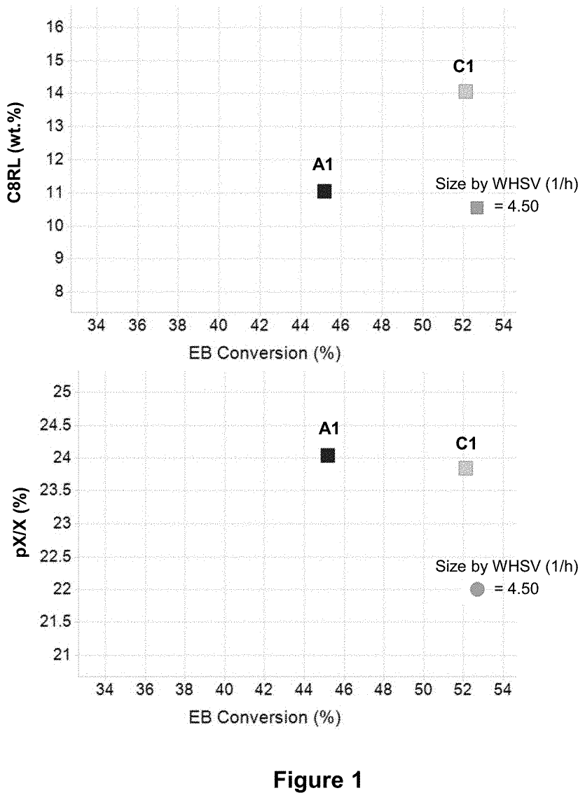

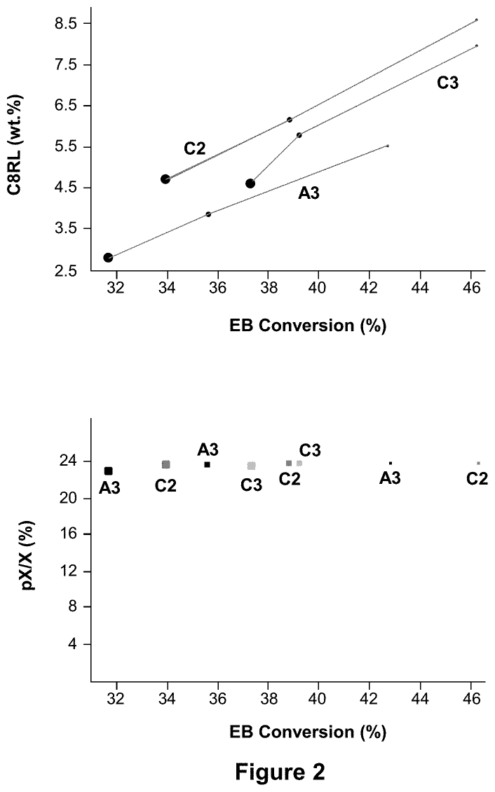

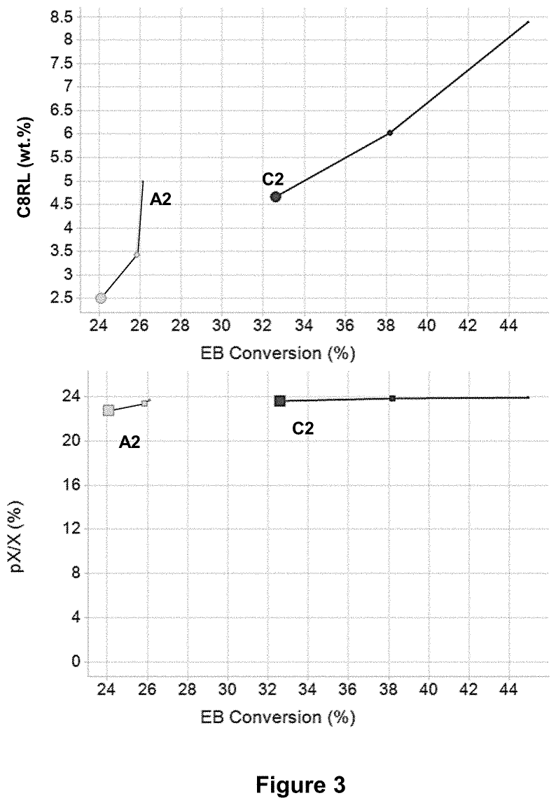

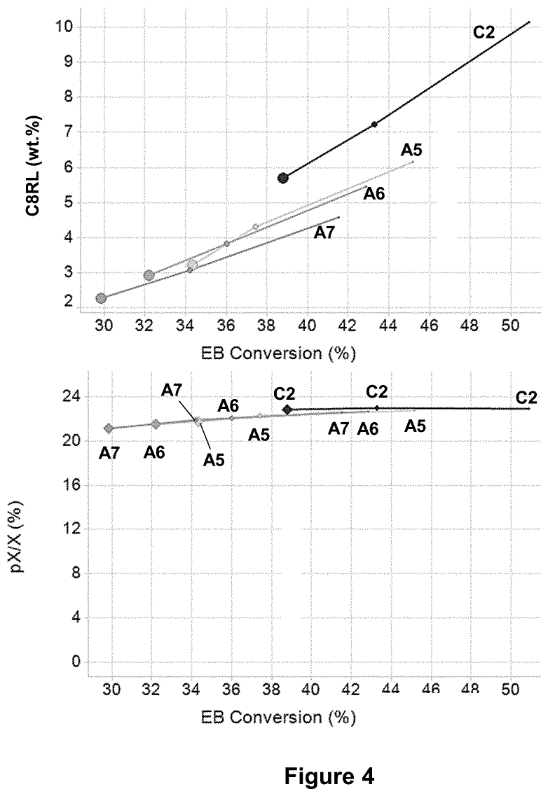

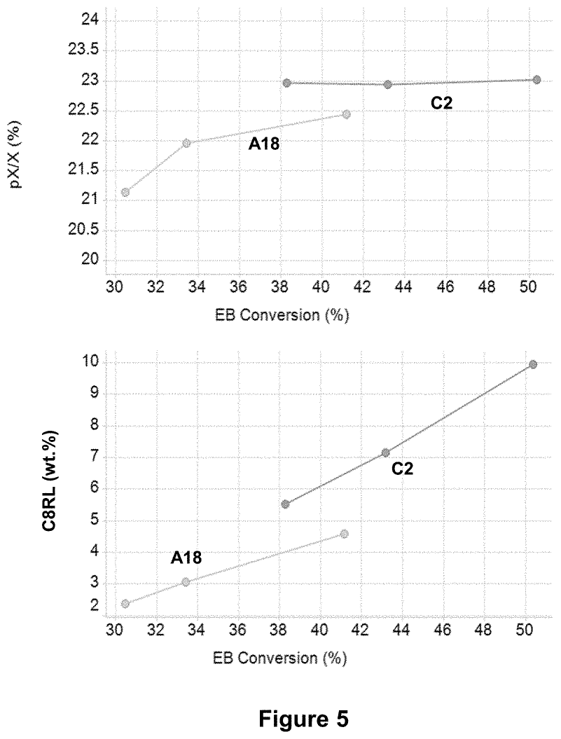

Catalysts prepared according to Example 1 were placed in a fixed-bed reactor and heated to 300° C. under a stream of pure H₂ (GHSV 800 h⁻¹; 4-8 bar). After 16 hr., a C₈ hydrocarbon feed comprising 10-20 wt. % ethylbenzene, 50-60 wt. % m-xylene, 10-25 wt. % o-xylene, 0-10 wt. % para-xylene, and 0-15 wt. % C₈ non-aromatics was introduced to the reactor at a weight hourly space velocity (WHSV) of 1-4 h⁻¹. The catalyst was increased to 360° C. and maintained at that temperature for 24-48 hr. before data collection. Results are provided in Table 2, below, and in FIGS. 1-5.

TABLE 2
Transalkylation Results

| | EB Conversion | pX/X | C8RL |
|---|---|---|---|
| Catalyst | (%) | (%) | (%) |
| C1 | 52.1 | 23.9 | 14.1 |
| C2 | 38.2 | 23.9 | 6 |
| C3 | 39.2 | 23.8 | 5.8 |
| C4 | 43.3 | 23 | 7.2 |
| C5 | 43.2 | 23 | 7.1 |
| A1 | 45.2 | 24 | 11.1 |
| A2 | 25.8 | 23.4 | 3.4 |
| A3 | 35.6 | 23.6 | 3.8 |
| A4 | 38.8 | 23.8 | 5.7 |
| A5 | 37.4 | 22.3 | 4.3 |
| A6 | 34.2 | 22 | 3.1 |
| A7 | 36.1 | 22.1 | 3.8 |
| A8 | 46.4 | 23.9 | 6.7 |
| A9 | 42.3 | 23.8 | 5.9 |
| A10 | 37.7 | 23.2 | 4.7 |
| A11 | 25.9 | 21.7 | 2.2 |
| A12 | 34.8 | 23 | 7.5 |
| A13 | 44.1 | 23 | 5.7 |
| A14 | 43.6 | 22.9 | 6.5 |
| A15 | 33 | 21.8 | 3.1 |
| A16 | 28 | 22.3 | 4.5 |
| A17 | 36.2 | 22.3 | 4.3 |
| A18 | 33.4 | 22 | 3.1 |
| A19 | 33.5 | 21.8 | 3.2 |
| A20 | 43.4 | 22.9 | 6 |
| A21 | 44.6 | 23 | 7.2 |
| A22 | 44.8 | 22.8 | 6.3 |

The results, provided in Table 2, above, and in FIGS. 1-5 demonstrate that inclusion of vanadium in the aromatic transalkylation catalyst appears to provide similar or improved ethylbenzene (EB) conversion to near-equilibrium levels of para-xylene (i.e., measured as a percentage of total xylenes (pX/X)), with improved selectivity (i.e., reduced C₈ ring loss (C8RL)).

1. A calcined aromatic transalkylation catalyst composition comprising:
vanadium, present in the composition in an amount within the range of 0.01 wt. % to 8 wt. %, calculated as elemental metal;
one or more zeolites, present in the composition in a combined amount within the range of 1 wt. % to 80 wt. %;
an inorganic binder, present in the composition in an amount within the range of 10 wt. % to 99 wt. %; and
a transition metal catalyst, present in the composition in an amount up to 8 wt. %, calculated as elemental metal.
